SEQUENCE LISTING (21 sequences)

SEQ ID NO: 1 - DNA, 2976 bp, Oryza sativa
cacggctgataaattagaagacatactatgagtccgtctttttctgtctgattccagaacaatcaatatctacatatgaatccatacattatctgctctgatttcgaaacataataatataggagaaaagaattaaaaatatgtgtaatcattattcgtcaaaatccgatatgtttcatagttccatacgtaattgcttggtttgacagtggtgaatcgatctagaaccttcgacaacaatcgagaacatatatggtcctcagatcttttgttaaggccgtgaaaacggaggtgattcgatgggaagacagacatgattgattcccatataggaataactgattcaagttctgaggtcaggccccccccattgcagatccatcagggaacctacctacagtattagctagacttgtgcaaagtaaaaccatggttattctgaatctaaacgatgcttcttcagagaatggttcagggcaccatatcaacttagcagatatcggatggtacacctgacctcaacatggggtgcttttgctactgcttgatcacagggtagggcctagcattatgccaggaaatgtagttagattcatacacacaaaagtgattaacaagaactacactagtggcctgaggagcccagacaaaagaagatcaataatcagcgaaggtaattatgccagcttctggatggatggtgcatctgtgattgattcactgatctgatcgctcactcgtcagctatcttggttccatgcctctcatgaaaagctaaggggttgcagagaagcgtggattttccctcttgtggcctggcctcatgccaatgcgctagtctcatctcaggcagcacaagctgtcttttcatcctgtagatcgtgcaaaataaggtgctggttctaaacgtcccccagaaagccctctctgtttgcacatgtgtgtatttagtggtatttttcagtaccaatatccatttattttcatttaatttgctttgctcgtaaagtagttcttgattctacatgtatacatctacaaagtattgatgagtgctcattacagaaggcatctcaaatcaataaattatctgcatttttgcgaaagaaacctgattgaaacacctcgtgaacgaaatacctagcaaactctgtaaggcctgagattttcaccaagtcgagtggctgatctgcaacgagctgtaccgatcaaaatatgggttctttcattctttgtgatgtgtgctgattttccaatcgaaaatcattgtggcaagattttgtcagggcatccccgtccacactctgctcccccaccggggatgcctaccaagggaagaagaggcgtcataactgccatacacttgtgctgtctacggccatcagagcattgaccatacgggcctacttcacagaacatgattgacctgtaaaaatcagcttcagactttgagttccgaatcctgttgatttttcattgagtttaattaggagtaggtggcattgctcttcagatgatatgtcgatttctggcattgctctttttaatacaaggtgatgaaaattcagctccctgaattggagttttgttttcctgaactgtagtatctcaactctgaagacagttactgatagtggtagtacaagatagtactccctccgttttgaaatgtttgacgccgttgactttttatcacatgtttgatcattcgtcttattcaaaaaatttaagtaattattaattattttcctatcatttgattcattattaaatatatttttatgtagacatataattttacatatctcacaaaagtttttgaataagacgaacagttaaacatgtgctaaaaagtcaacggtgtcaaacatttcgaactggagggagtatcctacaggtacagtacggcaaaaaaagaaaaactgaatgtgagctaagctcaatgagagaagctaggattgcaaattgctgaagtactccaactgacatgagatttttcaatagtagcaggtcagttttgacagtgaccatccaagtgcaacgtcctctgctctgacattgcttagcattgctaaccgaagcatgcacactgcgtaatagagtggttaggataaccccttattgtaatgtcacctttgcaaatccttaactgctcggatatttcaatttggtcaccagagatggcaatcctacaattgaaaatttgttcagttcccacggatccatcattaatctggcaatggcggcaacctctgacagggacaatggcaaattcggccaatagtaaatttccgtacggtttatcctagttggcattggcacacatggttcgtctcttctacgagtatagattatgaaaaatgtcaacttacaacaggtgacgaatttcgcaaaaaaaacgtattaacattcggcatggaaaacgtacgtagaatgaccaaaaatatccatccctatagtatcatttctttcaggggagcccccaatctacaaaagaaaaagaatttgttcgtcacccatatatccccgtcatgacctcgacgtcccgctttatccaggcatatagtttacaacaccttgtgaattgaaaacccacaattatttcagtctaacagcagacagaggcaacgttgctctcgttgtcgttcacggggggatgacgcgcggttttatgccctcgacgagaatacaaaatcaagtatgcgtttctgtttctcggccaatgctgatccgacaacgtgtttgaacggattaaacaaaatctgaatccccgtcgaaaaattagaccagaaacaatgatcttatgctgattaattagggctaatgagctatgcatgcaagcactgtacccagtggtgctccgacaagtaggcctgcctaatcaaaaggcagtgaggactgtaactactagtacctgccacctcccagttgctcaggcttctcaaccttagctagctcgatctccctataaatact

SEQ ID NO: 2 - DNA, 300 bp, Unknown
  computer assisted result identifying regulatory elements of interest from Zea mays
atgagcaccagaaccgaacactgctcagagttccaagacaaggtgtcccggcccaatgagtcgcctgcaactgtaatcgagtggttgggcttgggcccgagggcctatcggccattcatcatcaccgtctctctttgcctgggccgctccaatgtgacatgacctgatgtgacgcgacgtgatacgatcccaccgcgcggcgcggagcacacgggtggctagtagtgtagtagggccggcagggcatcttttctgtgggcctgtggctggtgcagggagagagatgaggtaccggcgctg

SEQ ID NO: 3 - DNA, 300 bp, Unknown
  computer assisted result for complementary sequence of SEQ ID NO2, identifying regulatory elements of interest from Zea mays
tactcgtggtcttggcttgtgacgagtctcaaggttctgttccacagggccgggttactcagcggacgttgacattagctcaccaacccgaacccgggctcccggatagccggtaagtagtagtggcagagagaaacggacccggcgaggttacactgtactggactacactgcgctgcactatgctagggtggcgcgccgcgcctcgtgtgcccaccgatcatcacatcatcccggccgtcccgtagaaaagacacccggacaccgaccacgtccctctctctactccatggccgcgac

SEQ ID NO: 4 - DNA, 3143 bp, Unknown
  computer assisted result from Oryza sativa
cacggctgataaattagaagacatactatgagtccgtctttttctgtctgattccagaacaatcaatatctacatatgaatccatacattatctgctctgatttagaaacataataatataggagaaaagaattaaaaatatgtgtaatcattattcgtcaaaatccgatatgtttcatagttccatacgtaattgattggtttgacagtggtgaatcgatctagaaccttcgacaacaatcgagaacatatatggtcctcagatcttttgttaaggccgtgaaaacggaggtgattcgatgggaagacagacatgattgattgccatataggaataactgattcaagttctgaggtcaggcgcgcgccattgcagatgcatcagggaacctacctacagtattagctagacttgtgcaaagtaaaaccatggttattctgaatctaaacgatgcttcttcagagaatggttcagggcaccatatcaacttagcagatatcggatggtacacctgacctcaacatggggtgcttttgctactgcttgatcacagggtagggcctagcattatgccaggaaatgtagttagattcatacacacaaaagtgattaacaagaactacactagtggcctgaggagcgcagacaaaagaagatcaataatcagcgaaggtaattatgccagcttctggatggatggtgcatctgtgattgattcactgatctgatcgctcactcgtcagctatcggttttccatgcctctcatgaaaagctaaggggttgcagagaagcgtggattttccctcttgtggcctggcctcatgccaatgcgctagtctcatctcaggcagcacaagctgtcttttcatcttgtagatcgtgcaaaataaggtgctggttctaaacgtgccccagaaagcgctctctgtttgcacagtgtaaagatttagtggtatttttcagtaccaatatccatttattttcatttaatttgctttgctcgtaaagtagttcttgattctacatgtatacatctacaaagtattgatgagtgctcattacagaaggcatttcaaatcaataaattatctgcatttttgcgaaagaaacctgattgaaacacctcgtgaacgaaatacctagcaaactctgtaaggcctgagattttcaccaagtcgagtggctgatctgcaacgagctgtaccgatcaaaatatgggttctttcattctttgtgatgtgtgctgattttccaatcgaaaatcattgtggcaagattttgtcagggcatcgccgtccacactctgctcccccaccggggatgcctaccaagggaagaagaggcgtcataactgccatacacttgtgctgtctacggccatcagagcattgaccatacgggcctacttcacagaacatgattgacctgtaaaaatcagcttcagactttgagttccgaatcctgttgatttttcattgagtttaattaggagtaggtggcattgctcttcagatgatatgtcgatttctggcattgctctttttaatacaaggtgatgaaaattcagctgcctgaattggagttttgttttcctgaactgtagtatctgaactctgaagacagttactgatagtggtagtacaagatagtactccctccgttttgaaatgtttgacgccgttgactttttatcacatgtttgatcattcgtcttattcaaaaaatttaagtaattattaattattttcctatcatttgattcattattaaatatatttttatgtagacatataattttacatatctcaaaaaagtttttgaataagacgaacagttaaacatgcgctaaaaagtcaacggtgtcaaacatttcgaactggagggagtatcctacaggtacagtacggcaaaaaaagaaacactgaatgtgagctaagctcaatgagagaagctaggattgcaaattggtgaagtactccaactgacatgagatttttcaagttagtagcaggtcagttttgacagtgaccatccaagtgcaacgtcctctgctctgacattgtttaggccttgctaaccgaagcatgcatactgcgtaagtgtagagtggttaggataaccccttattgtaatgtcacctttgcaaatacttaactgctcggatatttcaatttggtcaccagagatggcaatccaaaatacaattgaaaatttgttcagttgccacggatccatcattaatctagcaatggcggcaacctctgacagggacaatggcaaattcggccaatagtaaatttcgatcctagttggcattggcacacatggttcgtctcttctacgagtatagattatgaaaaatgtcaacttacaacaggtgacgaatttcgcaaaaaaaacgtattaacattcggcatggaaaacgtacgtagaatgaccaaaaatatccatccctatagtatcatttctttcaggggagcccccaatctacaaaagaaaacggatttaaaccaagttcgtcacccatatatcggcgtcatgacctcgacgtcgcgctttatccaggcatatagtttacaacaccttgtgaattgaaaacccacaattagtgtatttcagtctaacagcagacagaggcaacgttgctctcgttgtcgttcacggggggatgacgcgcggttttatgccctcgacgagaatacaaaatcaagtatgcgtttctgtttctcggccaaatgctgatccgacaaggtgtttgaacggattaaacaaaatctgaatccccgtcgaaaaattagaccagaaacaatgatcttatgctgattaattagggctaatgagctatgcatgctatgcctgtacccagtggtgaaagtctccgacaagtaggcctgcctaatcaggaggtagtgaggactgtaactactagtacctgacacctcccagttgctcaggcttctcaaccttagatagctagatctccctataaatactcctgctcattaccacaacgtgcgcgtgcaaccgatcgacggagcgagcgagctagccagccagtgttagagcttgagctgcttgttcttcttctacctcctgcactcgcgtgctgcacaagtagctcagctagatagagcgtcagaa

SEQ ID NO: 5 - DNA, 384 bp, Unknown
  computer assisted result from Zea mays
tttgcacccgtaattcaacggacgcattattcaccgcctgacagataggctagcttctaggtcaaaaaccagcaaggttctcgcaatcaagcatccgctatcgcatgtcaacctctctgtctcatgtcgacattgctcacaccctctcgtggctctgagaatcatatacgcctacgcagctatggcgacggctggggcctcagggtatgcagtggccaacatgtacagatgcttctactgctgatcacttactaataatctaaacccaaaagaagttaataggcacggtgatgggactgatcacttactagctcagttagtacagggtgctaaggaggtgtggaccggagcaccatgcacgaccagctgctggccaggcccgaa

SEQ ID NO: 6 - DNA, 13 bp, Unknown
  output identifying the regulatory motifs identified through comparisons of ADF4 promoters from maize, sorghum, and rice
gcgtcatgacctc

SEQ ID NO: 7 - DNA, 13 bp, Unknown
  output identifying the regulatory motifs identified through comparisons of ADF4 promoters from maize, sorghum, and rice
tccgacaagtagg

SEQ ID NO: 8 - DNA, 13 bp, Unknown
  output identifying the regulatory motifs identified through comparisons of ADF4 promoters from maize, sorghum, and rice
tttgttcgtcacc

SEQ ID NO: 9 - DNA, 12 bp, Unknown
  output identifying the regulatory motifs identified through comparisons of ADF4 promoters from maize, sorghum, and rice
ccctataaatac

SEQ ID NO: 10 - DNA, 10 bp, Unknown
  output identifying the regulatory motifs identified through comparisons of ADF4 promoters from maize, sorghum, and rice
agagtggtta

SEQ ID NO: 11 - DNA, 10 bp, Unknown
  output identifying the regulatory motifs identified through comparisons of ADF4 promoters from maize, sorghum, and rice
caattgaaaa

SEQ ID NO: 12 - DNA, 13 bp, Unknown
  output identifying the regulatory motifs identified through comparisons of ADF4 promoters from maize, sorghum, and rice
agaatttgttcgt

SEQ ID NO: 13 - DNA, 30 bp, Unknown
  output identifying the regulatory motifs identified through comparisons of ADF4 promoters from maize, sorghum, and rice
gcgtcatgacctcgacgtcgcgctttatcc

SEQ ID NO: 14 - DNA, 28 bp, Unknown
  promoter element from meristematic tissue matching TGGGCC
cgaggtgggcccgtaggtgggcccgtat

SEQ ID NO: 15 - DNA, 24 bp, Unknown
  promoter element of stem element 1 (SE1)  from bean matching TGGGCC
ataatgggccacactgtggggcat

SEQ ID NO: 16 - DNA, 11 bp, Unknown
  promoter element of a light responsive element (L-box) matching TCCCAC
atcccacctac

SEQ ID NO: 17 - DNA, 21 bp, Unknown
  promoter element from seed storage protein napA matching TCCCAC
gatcccacatacacatacacg

SEQ ID NO: 18 - DNA, 30 bp, Unknown; identical to SEQ ID NO: 13
  promoter elements from Zea mays

SEQ ID NO: 19 - DNA, 492 bp, Unknown
  sequence showing three promoter elements from Zea mays
accaggattggacttgaggcacttagccttgaagactggttcgaagaaccagaacccgatccacctgacccagtggaccgccagaagatagaagatatcctggacctgcaagatgtcagcaatgacgattgaaagattcccaggatagccggcggacgtggtggacccagtctaggtgcgatgcttagtcacgcacgatgactctgtcggaaggcatctttactttcggcaaactttaataatactttaggaaaagtattgtacaagttaggtgcagaatcaataatgcacccagctttagtcttgtctactgaattattgtgtcggttgcattattggatgcctgcgtgcaccctaagcaatccccggctctcatctctataagaggagcctttgtattcagttgcaagcatgcaagtcacacactgcaagcttacttctgagcaaaaagagttttgagtgaaataaatttgaagttcccccttacatctt

SEQ ID NO: 20 - DNA, 492 bp, Unknown
  a tetracycline regulated BSV promoter
accaggattggacttgaggcacttagccttgaagactggttcgaagaaccagaacccgatccacctgacccagtggaccgccagaagatagaagatatcctggacctgcaagatgtcagcaatgacgattgaaagattcccaggatagccggcggacgtggtggacccagtctaggtgcgatgcttagtcacgcacgatgactctgtcggaaggcatctttactttcggcaaactttaataatactttaggaaaagtattgtacaagttaggtgcagaatcaataatgcacccagctttagtcttgtctactgaattattgtgtcggttgcattattggatgcctgcgtgcaccctaactctatcagtgatagagtctctataagactctatcagtgatagagttgcaaactctatcagtgatagagtcaagcttacttctgagcaaaaagagttttgagtgaaataaatttgaagttcccccttacatctt

SEQ ID NO: 21 - DNA, 10 bp, Unknown
  exemplary TATA box containing sequence
tctcrataag

(19) United States
(12) Patent Application Publication
(10) Pub. No.: US 2010/0138952 A1
(43) Pub. Date: Jun. 3, 2010

(54) GENE PROMOTER REGULATORY ELEMENT ANALYSIS COMPUTATIONAL METHODS AND THEIR USE IN TRANSGENIC APPLICATIONS

(75) Inventors: CARL R. SIMMONS, DES MOINES, IA (US); PEDRO A. NAVARRO ACEVEDO, ANKENY, IA (US)

Correspondence Address: MCKEE, VOORHEES & SEASE, P.L.C.;ATTN: PIONEER HI-BRED, 801 GRAND AVENUE, SUITE 3200, DES MOINES, IA, 50309-2721, US

(73) Assignee: PIONEER HI-BRED INTERNATIONAL, INC., Johnston, IA (US)

(21) Appl. No.: 12/534,471

(22) Filed: Aug. 3, 2009

Related U.S. Application Data
(60) Provisional application No. 61/086,372, filed on Aug. 5, 2008.

(51) Int. Cl. C12N 15/82; G06N 5/02

(52) U.S. Cl. 800/278; 435/320.1; 706/54

(57) ABSTRACT

A computer-assisted method of identifying regulatory elements includes receiving a first orthologous species sequence, receiving a word length, receiving a relative offset, and receiving at least one additional orthologous species sequences, wherein each of the orthologous species sequences is associated with a species, and each of the species is an orthologous species. The method further includes performing a pairwise comparison between each pair of orthologous species sequences, computing using a computing device, overlapping portions of the sequence overlapping the sequences of all of the orthologous species sequences within the relative offset and greater than or equal to the word length.

The method further includes providing an output to a user identifying the overlapping portions of the sequence for all of the orthologous species sequences to identify candidate regulatory elements.

### CROSS-REFERENCE TO RELATED APPLICATIONS

This application claims priority under 35 U.S.C. §119(e) to provisional application Ser. No. 61/086,372 filed Aug. 5, 2008 herein incorporated by reference in its entirety.

### FIELD OF THE INVENTION

The present invention relates to the field of plant molecular biology and plant genetic engineering and more specifically relates to polynucleotide molecules useful for control of gene expression in plants and the identification of candidate gene promoter regulatory elements using bioinformatics.

### BACKGROUND OF THE INVENTION

One of the goals of plant genetic engineering is to produce plants with desirable characteristics or traits. Technological advances have provided the requisite tools to transform plants to contain and express foreign genes. The technological advances in plant transformation and regeneration have enabled researchers to take an exogenous polynucleotide molecule, such as a gene from a heterologous or native source, and incorporate that polynucleotide molecule into a plant genome. The gene can then be expressed in a plant cell to exhibit the added characteristic or trait. In one approach, expression of a gene in a plant cell or a plant tissue that does not normally express such a gene may confer a desirable phenotypic effect. In another approach, transcription of a gene or part of a gene in an antisense orientation may produce a desirable effect by preventing or inhibiting expression of an endogenous gene.

Expression of heterologous DNA sequences in a plant host is dependent upon the presence of an operably linked promoter that is functional within the plant host. Choice of the promoter sequence will determine temporal and spatial expression within the organism the heterologous DNA sequence is expressed. Thus, where expression is desired in a preferred tissue of a plant, tissue-preferred promoters are utilized. In contrast, where gene expression throughout the cells of a plant is desired, constitutive promoters are preferred. Additional regulatory sequences upstream and/or downstream from the core promoter sequence may be included in expression constructs of transformation vectors to bring about varying levels of tissue-preferred or constitutive expression of heterologous nucleotide sequences in a transgenic plant. Isolation and characterization of promoters and terminators that can serve as regulatory elements for expression of isolated nucleotide sequences of interest in are needed for impacting various traits in plants.

Numerous promoters, which are active in plant cells, have been described in the literature. These promoters and numerous others have been used in the creation of constructs for transgene expression in plants. Despite the number of promoters, there is still a need for novel promoters and regulatory elements with beneficial expression characteristics.

For production of transgenic plants with various desired characteristics, it would be advantageous to have a variety of promoters to provide gene expression such that a gene is transcribed efficiently in the amount necessary to produce the desired effect. The commercial development of genetically improved germplasm has also advanced to the stage of introducing multiple traits into crop plants, often referred to as a gene stacking approach. In this approach, multiple genes conferring different characteristics of interest can be introduced into a plant. It is often desired when introducing multiple genes into a plant that each gene is modulated or controlled for optimal expression, leading to a requirement for diverse regulatory elements. In light of these and other considerations, it is apparent that optimal control of gene expression and regulatory element diversity are important in plant biotechnology.

### BRIEF DESCRIPTION OF THE FIGURES

FIG. 1A is a block diagram of one system where a software application is accessible over a network.

FIG. 1B is a block diagram of another system where a software application resides on a computing device.

FIG. 2A is a representation of an input screen display.

FIGS. 2B and 2C additional representations of an input screen display.

FIG. 3 is a flow diagram of one methodology.

FIG. 4A is a representation of an output screen display identifying regulatory elements of interest.

FIG. 4B is a representation of another output screen display identifying regulatory elements of interest.

FIG. 5A is a representation of an output identifying the regulatory motifs identified through the method applied to comparisons of ADF4 promoters from maize, sorghum, and rice.

FIG. 5B is another representation of an output identifying the regulatory motifs identified through the method applied to comparisons from maize, sorghum, and rice.

FIG. 6 is a table illustrating promoter elements matching TGGGCC.

FIG. 7 is a table illustrating promoter elements matching TCCCAC.

FIG. 8 is a screen display illustrating promoter elements.

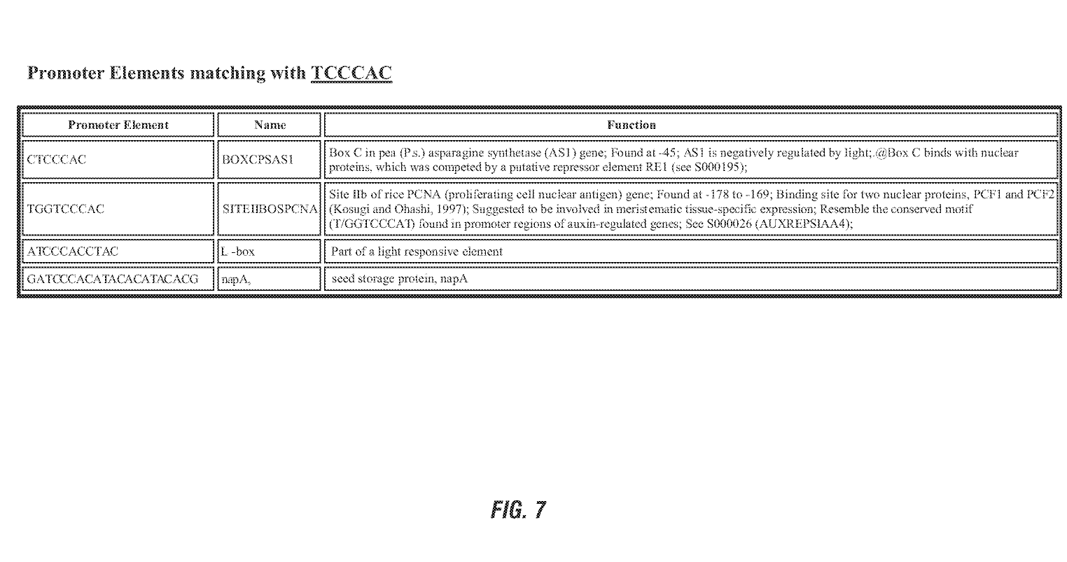

FIG. 9A illustrates the three promoter elements identified though the use of the method.

FIG. 9B is a tetracycline regulated BSV promoter engineered through the use of the method.

### SUMMARY

According to one aspect, a computer-assisted method of identifying regulatory elements includes receiving a first orthologous species sequence, receiving a word length, receiving a relative offset, and receiving at least one additional orthologous species sequence, wherein each of the orthologous species sequences is associated with a species, and each of the species is an orthologous species. The method further includes performing a pairwise comparison between each pair of orthologous species sequences, computing using a computing device, overlapping portions of the sequence overlapping the sequences of all of the orthologous species sequences within the relative offset and greater than or equal to the word length. The method further includes providing an output to a user identifying the overlapping portions of the sequence for all of the orthologous species sequences to identify candidate regulatory elements.

According to another aspect, a system for identifying regulatory elements includes a computer and an article of software executing on the computer. The article of software is adapted for performing steps of receiving a first orthologous species sequence, receiving a word length, receiving a relative offset, receiving at least one additional orthologous species sequence, wherein each of the orthologous species sequences is associated with a species, and each of the species is an orthologous species. The article of software is further adapted for performing a pairwise comparison between each pair of orthologous species sequences, computing overlapping portions of the sequence overlapping the sequences of all of the orthologous species sequences within the relative offset and greater than or equal to the word length, and providing an output to a user identifying the overlapping portions of the sequence for all of the orthologous species sequences to identify candidate regulatory elements.

According to another aspect of the present invention, a computer-assisted method of identifying regulatory elements is provided. The method includes receiving a first sequence;

receiving a word length, receiving a relative offset, receiving at least one additional sequence, performing a pairwise comparison between each pair of sequences, computing using a computing device, overlapping portions of the first sequence overlapping the sequences of all of the sequences within the relative offset and greater than or equal to the word length, and providing an output to a user identifying the overlapping portions of the first sequence for all sequences to identify candidate regulatory elements.

### DETAILED DESCRIPTION OF THE INVENTION

The following description is merely exemplary in nature and is in no way intended to limit the methods, their application, or uses.

As used herein, the term “orthologs” may refer to two genes of different species that share a common evolutionary ancestry. They can be derived from a speciation event and belong to different species.

As used herein, the term “orthologous” may refer to two or more species that share a common evolutionary ancestry.

As used herein, the term “regulatory element” may refer to intended sequences responsible expression of the associated coding sequence including, but not limited to, promoters, terminators, enhancers, introns, and the like. A “regulatory element” may be in different portions of the gene.

As used herein, the term “promoter” may refer to a regulatory region of DNA capable of regulating the transcription of a linked sequence. It may, but need not include a TATA box capable of directing RNA polymerase II to initiate RNA synthesis at the appropriate transcription initiation site for a particular coding sequence. A promoter may also include other recognition sequences generally positioned upstream or 5′ to the TATA box, which may be referred to as upstream promoter elements.

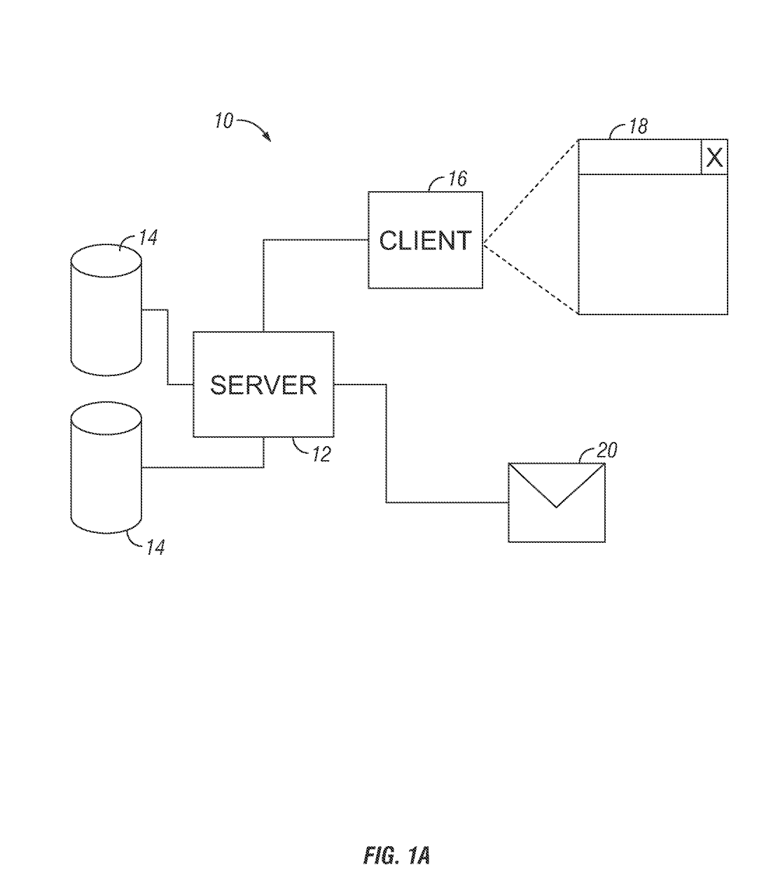

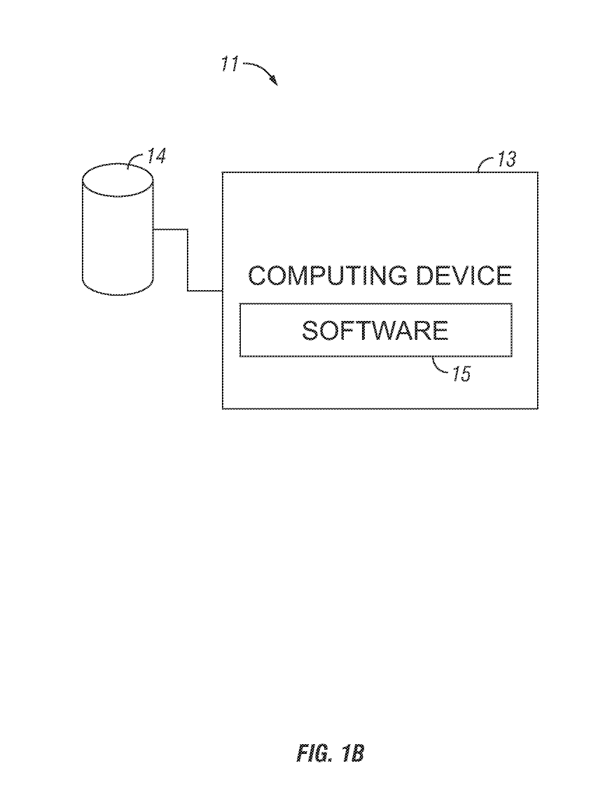

FIG. 1A illustrates a system for identifying regulatory elements. In the system shown, client computers access such as through use of a common web browser. The application may be implemented in any number of languages or software applications, including Java and perl. It is to be appreciated that due to the amount of processing required the results may be compiled and then emailed to users of the system. As shown in FIG. 1A, a system 10 includes a server 10 which is a computing device which has a computer readable medium associated therewith upon which software applications may be stored. One or more databases 14 may be in operative communication with the server 12. The one or more databases 14 contain data regarding various species of biological organisms. The databases 14 may be stored locally or be remotely accessible over a network. The server 12 is also in operative communication with one or more client computers 16. The client computers may access a software application residing on the server 12 in order to specify requests for identifying regulatory elements or receive the results of the requests for identifying regulatory elements. In the system 10 shown, a web browser 18 may be used on a client computer to make a request. The result of a request may be output to the web browser, or an email 20 may be sent to a user making the request due to the amount of processing required.

FIG. 1B illustrates another example of a system. In FIG. 1B, a system 11 include a computing device 13. A software application 15 executes on the computing device 13 to perform the methodology for identifying regulatory elements. The software application 15 may be written in the C# programming language and be run as a MICROSOFT WINDOWS desktop application. The software application 15 may be stored on a computer readable medium which is accessible by the computing device 13. A promoter element database 14 may also be stored locally on a computer readable medium which is accessible by the computing device 13. Thus, no network need be used.

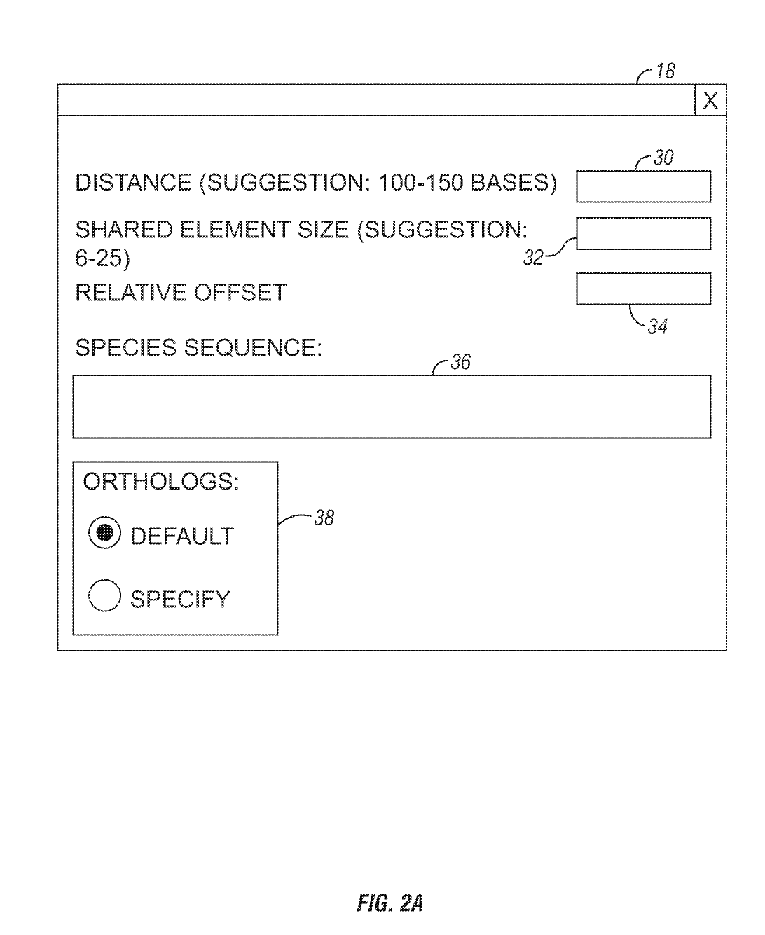

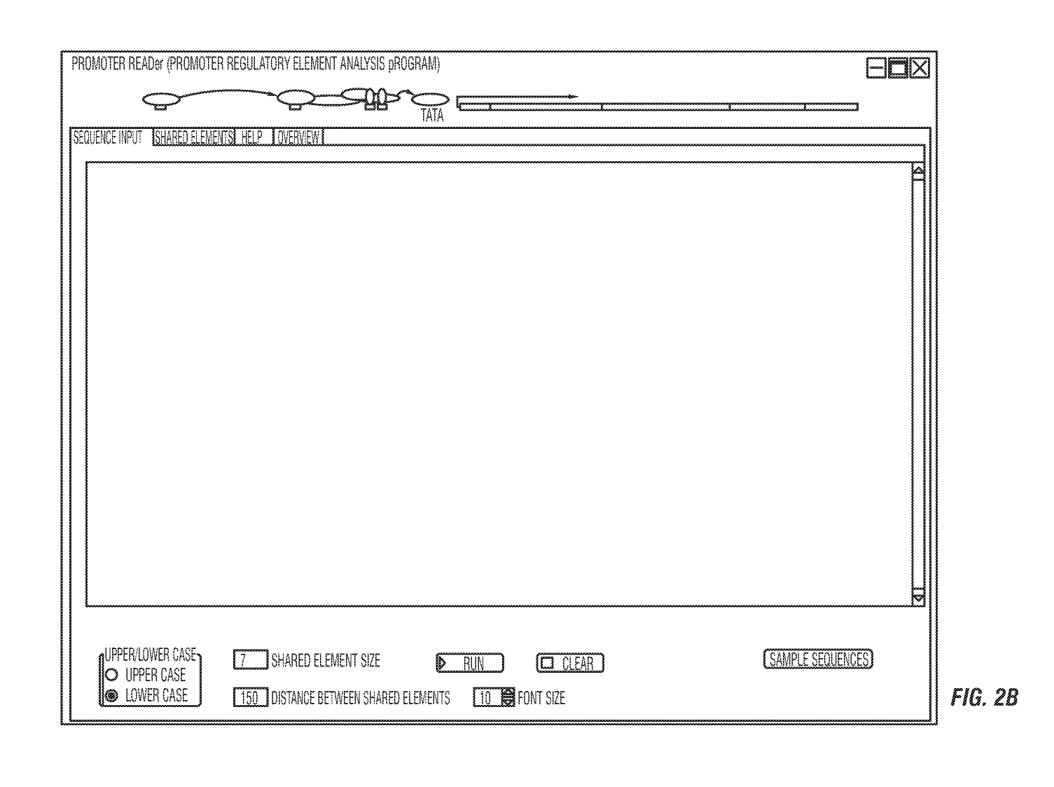

FIG. 2A shows an illustration of a screen display which may displayed on a display associated with a computer used by the user and allows a user to set various parameters. For example, the user can set a distance and a shared element size. Different results may be obtained where shared element sizes and distances and differ. As shown in FIG. 2A, a user may use the user interface shown in FIG. 2A to set various parameters. For example, the user may input a distance in the distance input box 30. Although a suggested distance of 100 to 150 bases is provided, more or fewer bases are permitted. The user may also input a shared element size in the shared element size input box 32. Although a suggested shared element size of 6 to 25 elements is provided, more or fewer elements are permitted. The user may also input a relative offset in the relative offset input box 34. In addition, the user may input the sequence of interest in the input box 36, such as by cutting and pasting the sequence from a file. Alternatively, a user could specify a file instead. As shown in FIG. 2A, a user may also specify orthologs if desired, or if not, default orthologs may be used.

FIG. 2B and FIG. 2C provide additional examples of a screen display which allows a user to set various parameters. In FIG. 2B, the screen display is shown before a sequence is input. FIG. 2C shows the screen display after a sequence is input.

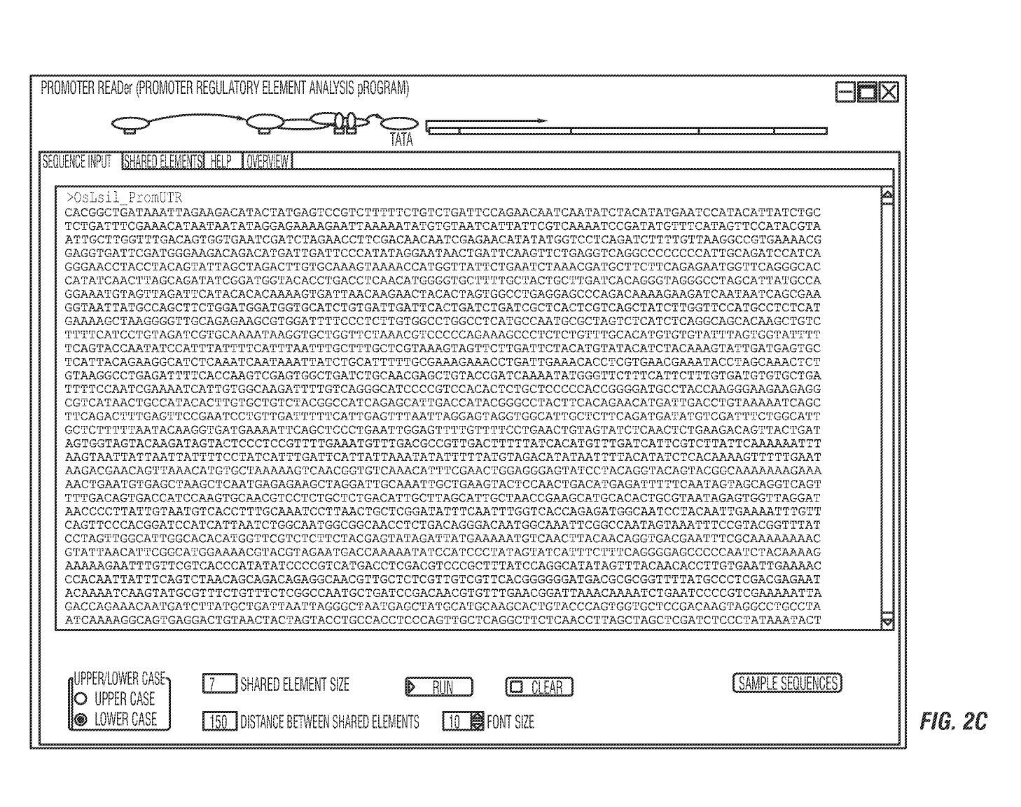

FIG. 3 illustrates one example of a methodology for comparison of three or more orthologous species. In step 40, a first orthologous species sequence is provided. In addition, the word length parameter is received in step 42 and the relative offset parameter is received in step 44. It is contemplated that defaults may be used for the parameters and the parameters may be specified in varying orders. Additional orthologous species sequences are received in step 46. A total of two of more orthologous species sequences should be used. Next in step 48, a pairwise comparison is performed between each pair of orthologous species sequences. In step 50 overlapping portions of the sequence overlapping all sequences are provided. In step 52, an output is provided. The methodology shown in FIG. 3 provides for comparison across three or more orthologous species. Different species may have genes that derived from a common ancestor. In addition to displaying sequence conservation, orthologs can frequently perform similar functions in different organisms. The phylogenetic relationship between the species may be taken into account when selecting the orthologous species from available sequenced orthologous species. One factor to consider is distinguishing conservation due to evolutionary proximity of species from conservation associated with regulatory elements of interest. Thus, the evolutionary proximity of at least one of the species should be sufficiently removed from the others to minimize or eliminate issues due to the evolutionary proximity of species. Another factor to consider is that it may be beneficial for one of the species to be significantly older than the other species.

It should be appreciated that confident identification of orthologs can also rely on the availability of suitability comprehensive collection of genes from both organisms. However, whether a particular set of species is appropriate can be readily determined from results obtained using the methodology. For example, if too many or too few candidate regulatory elements are consistently found, then it is apparent changes in the orthologous species used should be adjusted.

Where a maize species is of interest and one wants to find a particular promoter within a sequence associated with the maize species, other species that may be used may include rice, maize, and sorghum. Alternatively another monocot may be used such as onion, barley, or wheat.

Given three orthologous species, species A, species B, and species C, three pairwise comparisons are performed, namely A and B, A and C, and B and C. A distance is defined by the user which is a relative distance to an ATG start site (where DNA is used).

Although distance is a matter of user preference, useful distances include those on the order of about 100 bases or 150 bases. Of course, lesser or greater distances may be used. A shared element size is also selected by the user. The shared element size is a minimum size of interest to the user. Although shared element size is a matter of user preference, usually the shared elements size is in the range of 6 to 25. Having a size of at least six reduces the likelihood of random occurrences, un-related to conservation. Having a shared element size too large may miss possible regulatory elements. It is to be appreciated that the shared element size is a minimum size of interest to the user, so providing a relatively small shared element size of 6 or 7 will still capture much larger regulatory expressions where present. If two or more common elements overlap each other in every sequence used in the comparison, they are merged into a single element. Thus, specifying a 6-letter word size can produce a 30-letter common element.

The pairwise comparisons performed take into account the distance specified by the user in determining relative similarity. Thus, for example, where a distance of 100 bases or more is specified, the first shared element size of species A is search for in the 100 bases of species. Lengths which are more than or equal to the minimum size of interest are maintained for each pairwise comparison. Only those stretches of sequences common to all of the pairwise comparisons are considered to be candidate regulation elements. It should be appreciated that this methodology preserves relative order and approximate spacing across the entire set of species. It should further be noted that this approach does not rely upon complex scoring or statistical methods for evaluating possible alignments between the sequences of the different species, and thus do not have the same types of limitations and issues associated with such systems. It is also observed that gains in performance can be made by implementing the method using a non-linear binary search instead of linear approach. This reduces processing time significantly.

In addition, it is contemplated that more nuanced pattern searches may be used in making comparisons. In particular, some of the ‘letters’ in a word may be variables. It is further contemplates the analysis need not only be performed on forward-written words. In particular, words can be implemented in both the forward as well as the reverse direction. Some regulatory elements, especially those with ‘enhancer-like’ function can work in both directions.

Once candidate regulatory elements have been identified, this information may be used in various applications. Such applications may be relevant to transgenic research, such as improvement of crop plants. The method may be used for defining the boundaries of functional promoters. This may simplify sub-cloning processes; focus the research on promoter regions more likely to yield the full and desired expression pattern. It also enables efficient us of cloning vector space; some cloning vectors become unstable with large inserts. This issue is particularly germane to transgenic stacking experiments, because with more gene constructs packed into the same vector, the risk of vector instability increases, and once in the plant there is added risk to transformation efficiency and stability.

Various methods are available for using candidate sequences. Functional fragments can be obtained by use of restriction enzymes to cleave naturally occurring regulatory element nucleotide sequences. Alternatively, such elements may be synthesized from the naturally occurring DNA sequence; or can be obtained through the use of PCR technology. See particularly, Mullis et al. (1987) Methods Enzymol. 155:335-350, and Erlich, ed. (1989) PCR Technology (Stockton Press, New York), all of which are herein incorporated by reference. Where transformation vectors are formed, activity can be measured by Northern blot analysis, reporter activity measurements when using transcriptional fusions, and the like. See, for example, Sambrook et al. (1989) Molecular Cloning: A Laboratory Manual (2nd ed. Cold Spring Harbor Laboratory, Cold Spring Harbor, N.Y.), herein incorporated by reference. Reporter genes can be included in the transformation vectors. Examples of suitable reporter genes known in the art can be found in, for example: Jefferson et al. (1991) in Plant Molecular Biology Manual, ed. Gelvin et al. (Kluwer Academic Publishers), pp. 1-33; DeWet et al. (1987) Mol. Cell. Biol. 7:725-737; Goff et al. (1990) EMBO J. 9:2517-2522; Kain et al. (1995) BioTechniques 19:650-655; and Chiu et al. (1996) Current Biology 6:325-330, all of which are incorporated by reference. Additional information regarding transformation may be found in Regeneration of plants after transformation: McCormick et al. (1986) Plant Cell Reports 5:81-84, herein incorporated by reference in its entirety.

It may also be desired that expression associated with the candidate regulatory elements identified be suppressed. Methods of co-suppression are known in the art and can be similarly applied. These methods involve the silencing of a targeted gene by spliced hairpin RNA's and similar methods also called RNA interference and promoter silencing (see Smith et al. (2000) Nature 407:319-320, Waterhouse and Helliwell (2003)) Nat. Rev. Genet. 4:29-38; Waterhouse et al. (1998) Proc. Natl. Acad. Sci. USA 95:13959-13964; Chuang and Meyerowitz (2000) Proc. Natl. Acad. Sci. USA 97:4985-4990; Stoutjesdijk et al. (2002) Plant Phystiol. 129:1723-1731; and Patent Application WO 99/53050; WO 99/49029; WO 99/61631; WO 00/49035 and U.S. Pat. No. 6,506,559.

Thus, it should be apparent that once candidate regulatory elements are found, various methods may be applied. On example of a promoter which has been identified using the software methodology described herein is disclosed in U.S. Provisional Patent Application No. 60/963,878, entitled A Plant Regulatory Region That Directs Transgene Expression in the Maternal and Supporting Tissue of Maize Ovules and Pollinated Kernels, filed Aug. 7, 2007, and herein incorporated by reference in its entirety. See also U.S. Published Patent Application No. 2009-0094713 herein incorporated by reference in its entirety. The Published Patent Application discloses compositions comprising nucleotide sequences for a reproductive-tissue-preferred and preferentially an immature-ear-preferred promoter region for an actin depolymerization factor (ADF) gene, more particularly, the ADF4 promoter. Regulatory motifs of about six or eight bases within the ADF4 promoter sequence were identified by comparison to upstream sequences from orthologous genes from sorghum and rice. The 1000 base pairs upstream of the ADF4 promoter, relative to the ATG start of translation, were compared to the 1000 base pairs upstream sequence of the orthologous rice and sorghum genes. The comparison was performed through performing pairwise comparisons of multiple regulatory sequences from a plurality of orthologous species, here maize, rice and sorghum, to identify the regulatory motifs.

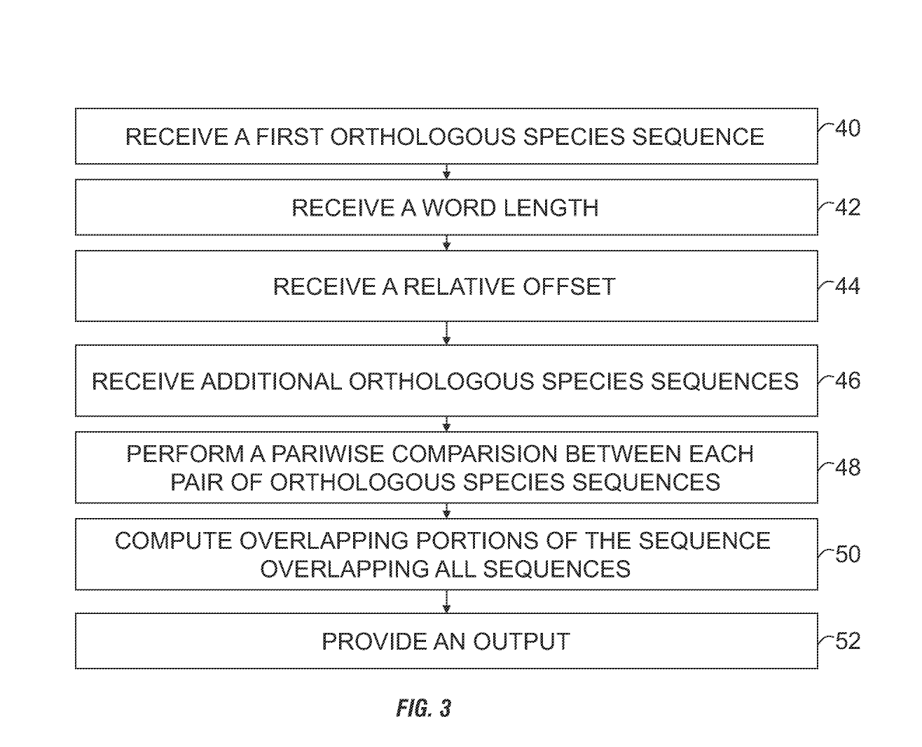

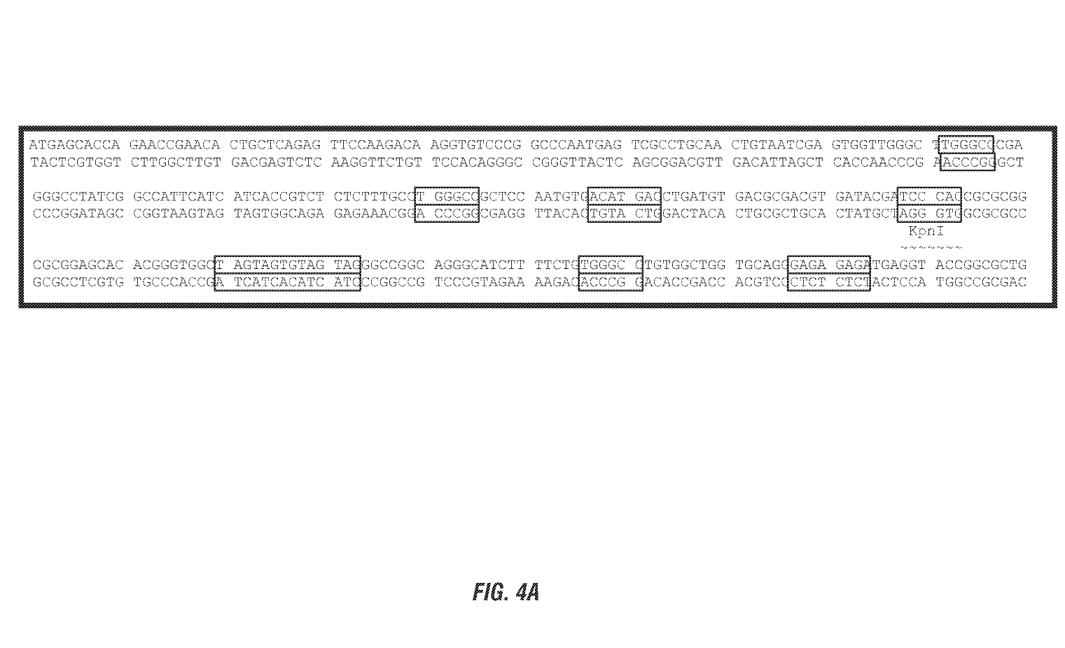

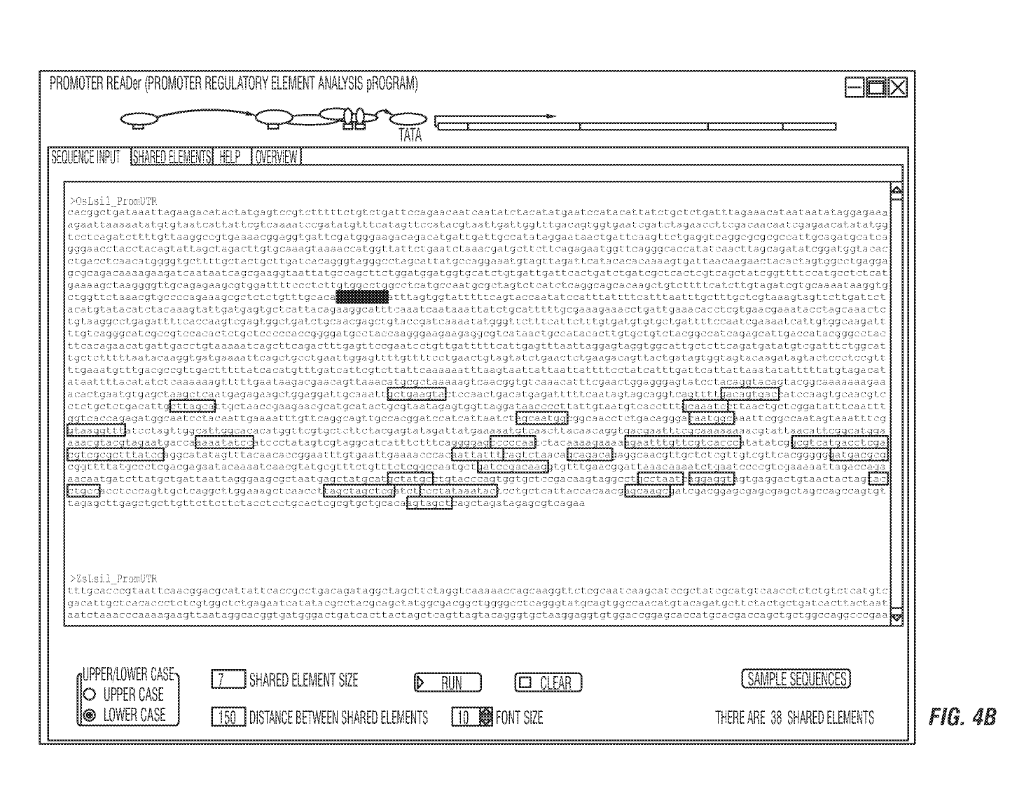

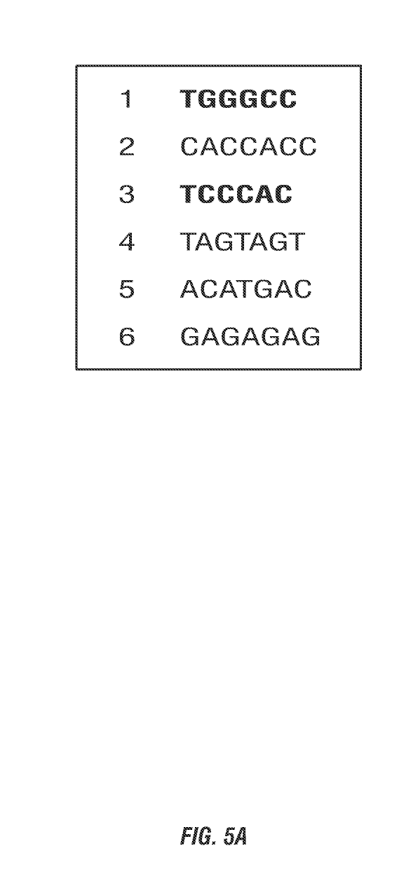

There the methodology and system described herein was applied to identify regulatory motifs in the ADF4 promoter. Regulatory motifs of about six or eight bases within the ADF4 promoter sequence were identified by comparison to upstream sequences from orthologous genes from sorghum and rice. The 1000 base pairs upstream of the ADF4 promoter, relative to the ATG start of translation were compared to the 1000 base pairs upstream sequence of the orthologous rice and sorghum genes to provide the output shown in FIG. 4A and FIG. 5A. FIG. 4A illustrates one example of results obtained. The results may be displayed on screen, printed, saved to a computer readable medium, emailed to a user or otherwise output. For the purposes of the trial shown in FIG. 4A, a gene from maize is used as the first orthologous species and a gene from rice and a gene from sorghum were used. A length of 6 was specified as well.

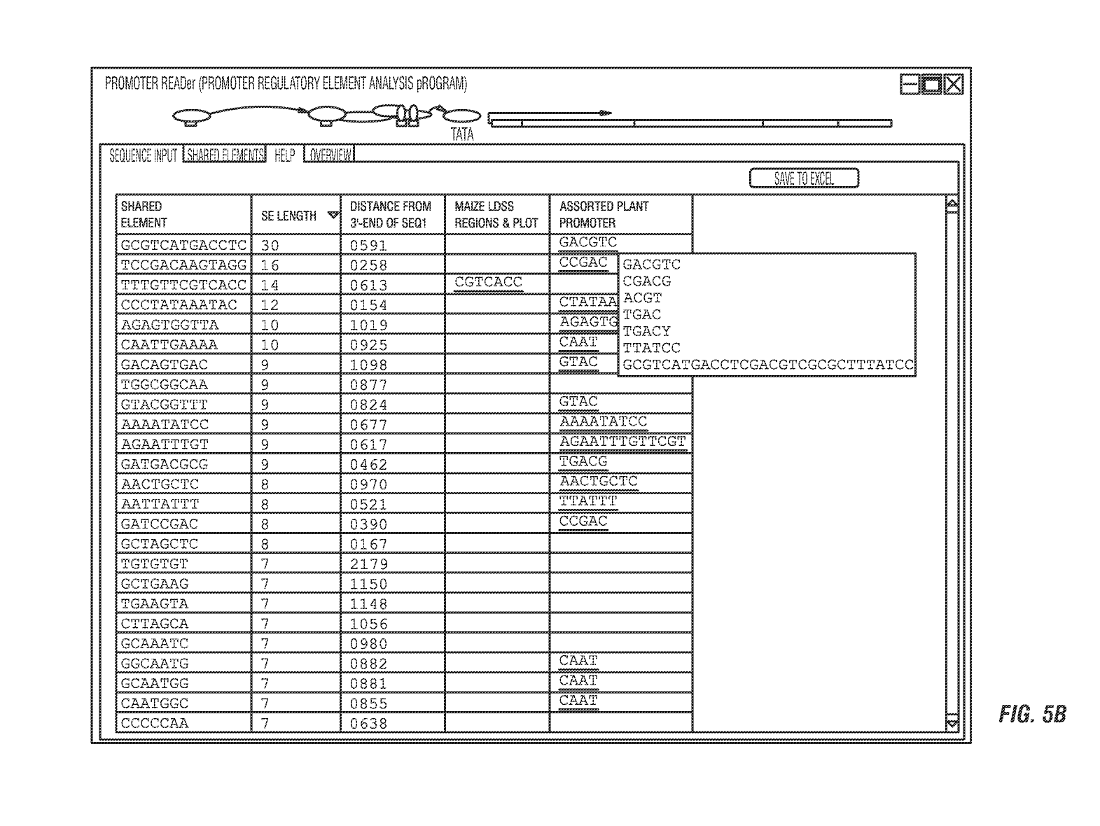

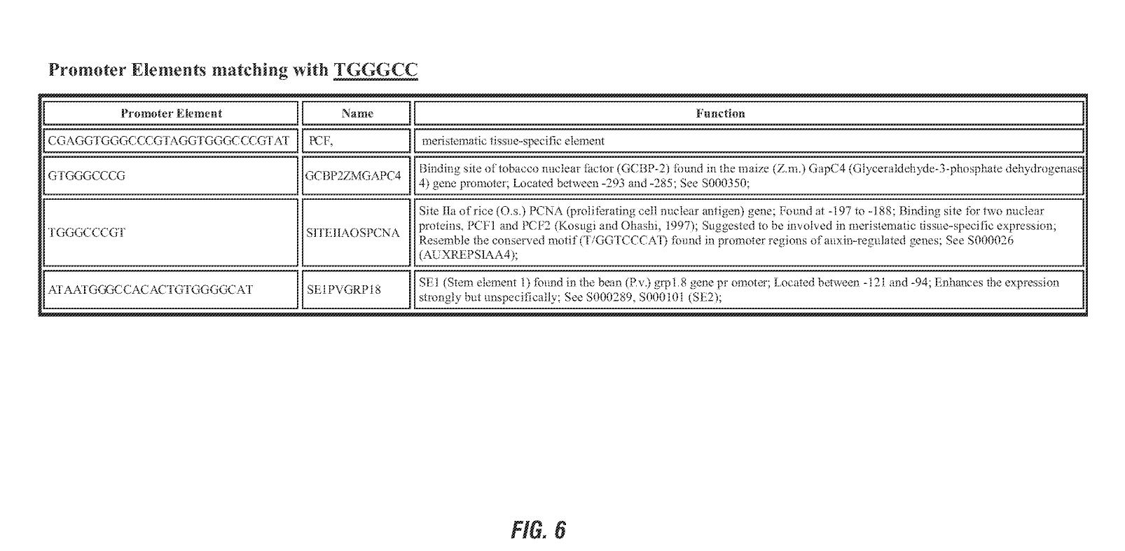

FIG. 5A identifies the regulatory motifs identified through the method applied to comparisons of ADF4 promoters from maize, sorghum, and rice. The result shown here is a listing of short promoter sequences that are preserved in the same relative order and approximate spacing across the set of promoters compared, and as well defines the likely promoter functional boundary. It is advantageous to have short promoter sequences because where large inserts are used in transgenic research there is generally increased risk of instability of the resulting cloning vector. The results obtained may also be advantageous due to the insight provided regarding the likely functional boundary. Because of the coalescing or growing of overlapping sequences, all sequences of the minimum size of interest or larger are identified. Thus, the method allows multiple promoters to be searched for simultaneously. In addition, the method assists in determining if upstream promoter sequences are present. Multiple trials may be performed with different lengths for the minimum size of interest or different distances for the same set of sequences. The use of multiple trials provides additional insight into regulatory elements of potential interest. FIG. 6 is a table illustrating promoter elements matching TGGGCC while FIG. 7 is a table illustrating promoter elements matching TCCCAC.

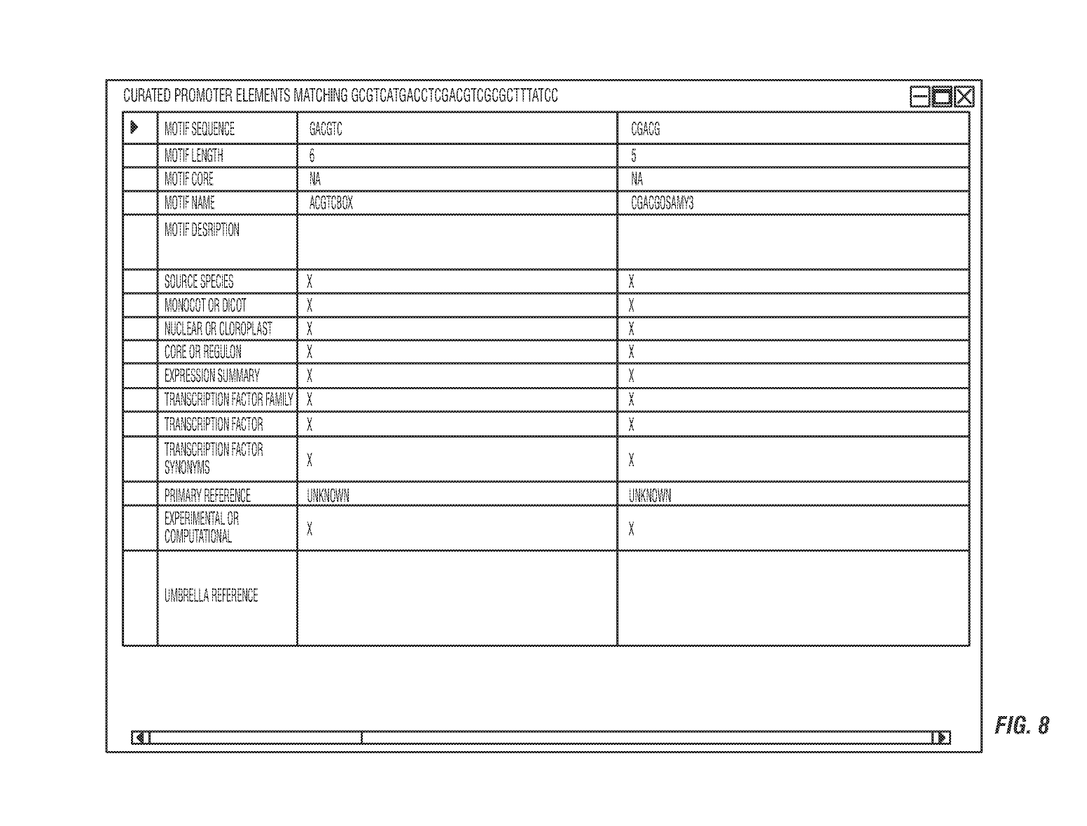

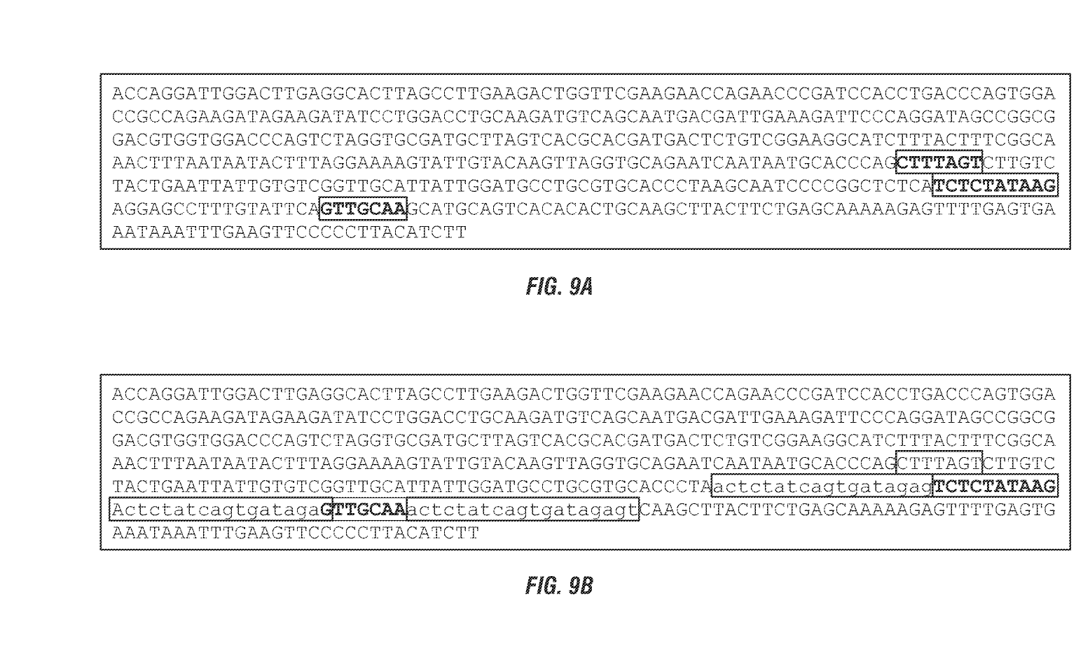

FIG. 9A and FIG. 9B provide an example of the use of the method to engineer a tetracycline regulated constitutive Banana Streak Virus (BSV) promoter. FIG. 9A illustrates the three conserved promoter elements identified through the method. Seven functional BSV promoters were compared with the method. The conserved regions identified are a putative TATA box, a conserved region near the putative start site, and a down stream conserved region. Note that when shown on a display associated with a computer, different colors may be used to identify different regions of interest. For example the TATA box (TCTCRATAAG) may be displayed in blue, the conserved region near the presumed start site (GTTGCAA) may be displayed in yellow, and other native conserved sites (CTTTAGT) may be displayed in gray.

FIG. 9B shows the placement of the three 19 nucleotide TetR sites. One is placed immediately upstream, and another is placed immediately downstream, of the TATA box site identified by the method. Note that when shown on a display associated with a computer, different colors may be used to identify different regions of interest. For example, the 19 nucleotide TetR site may be displayed in green. It will be appreciated that the gap between the TATA box and the GTTGCAA conserved site is 17 nucleotides. However, the last base of the TetR site is a “G”, so this can overlap with the GTTGCAA site. Also the first base of the TetR site is an “A”, which matches the native site. The third site is placed further downstream from the TATA box. Results from performing the methodology of the present invention have been used in engineering a tetracycline regulated constitutive Banana Streak Virus (BSV) promoter. Of course, the process may be applied for any number of specific purposes.

It should be appreciated that the methodology described does not require complex scoring rules such as may be associated with other methodologies. The process allows users to identify conserved candidate regulatory elements in gene promoters. Multiple promoters can be compared. The main approach is to compare promoters for orthologous genes across species, such as maize, rice and sorghum, or to compare genes within and/or between species that share expression patterns. The result is a listing of short promoter sequences that are preserved in the same relative order and approximate spacing across the set of promoters compared, and as well defines the likely promoter functional boundary.

The method may be used in various applications. Such applications may be relevant to transgenic research, such as improvement of crop plants. The method may be used for defining the boundaries of functional promoters. This may simplify sub-cloning processes and focus the research on promoter regions more likely to yield the full and desired expression pattern. It also enables efficient us of cloning vector space; some cloning vectors become unstable with large inserts. This issue is germane to transgenic stacking experiments, because with more gene constructs packed into the same vector, the risk of vector instability increases, and once in the plant there is added risk to transformation efficiency and stability. By allowing less DNA to be used, there is the practical advantage of having to describe and account for less introduced DNA, often a regulatory concern.

These methods allow identification of novel regulatory elements which may be novel and which alone or in combination may lead to methods for novel recombined or synthethic promoters having enhanced or novel expression capability. It should also be clear that multiple promoters may be searched for simultaneously. It should be appreciated that the methods may be used for comparing promoters and related types of diffuse regulatory elements, not necessarily promoters, and may be used for any organism, not just plants.

In addition, although discussed in the context of a comparative genomics method, sets of co-regulated genes (similar mRNA expression patterns), such as those of a common biochemical or signaling pathway may be used. These genes, from one or multiple species, also may serve as inputs to the program.

Although various specific embodiments and examples are provided herein, it should be understood that such examples and specific disclosure, while indicating embodiments of the invention, are given by way of illustration only. From the above discussion, one skilled in the art can ascertain the essential characteristics of the embodiments, and without departing from the spirit and scope thereof, can make various changes and modifications of them to adapt to various usages, conditions, and environments. Thus, various modifications of the embodiments in addition to those shown and described herein will be apparent to those skilled in the art from the foregoing description. Such modifications are also intended to fall within the scope of the appended claims.

What is claimed is:

1. A computer-assisted method of identifying regulatory elements, comprising:
receiving a first orthologous species sequence;
receiving a word length;
receiving a relative offset;
receiving at least one additional orthologous species sequences, wherein each of the orthologous species sequences is associated with a species, and each of the species is an orthologous species;
performing a pairwise comparison between each pair of orthologous species sequences;
computing using a computing device, overlapping portions of the sequence overlapping the sequences of all of the orthologous species sequences within the relative offset and greater than or equal to the word length;
providing an output to a user identifying the overlapping portions of the sequence for all of the orthologous species sequences to identify candidate regulatory elements.

2. The computer-assisted method of claim 1 wherein the candidate regulatory elements comprises a plurality of promoters.

3. The computer-assisted method of claim 1 further comprising constructing a transformation vector comprising at least one of the candidate regulatory elements.

4. The computer-assisted method of claim 3 further comprising producing a transgenic organism expressing the transformation vector.

5. The computer-assisted method of claim 1 further comprising using one or more candidate regulatory elements in a plant breeding program.

6. The computer-assisted method of claim 1 wherein the step of receiving the word length comprises receiving a user-specified word length through a user interface.

7. The computer-assisted method of claim 1 wherein the step of receiving the first orthologous species sequence comprises receiving a user-specified first orthologous species sequence through a user interface.

8. The computer-assisted method of claim 1 wherein the step of receiving the relative offset comprises receiving a user-specified relative offset through a user interface.

9. The computer-assisted method of claim 1 wherein the step of receiving the at least one additional orthologous species sequences includes receiving the at least one additional orthologous species from a database.

10. The computer-assisted method of claim 1 wherein the first orthologous species sequence and the at least one additional orthologous species sequences are associated with plants.

11. The computer assisted method of claim 10 wherein one of the first orthologous species sequence and the at least one additional orthologous species sequences is associated with maize.

12. The computer assisted method of claim 10 wherein one of the first orthologous species sequence and the at least one additional orthologous species sequences is associated with soybeans.

13. The computer assisted method of claim 10 wherein one of the first orthologous species sequence and the at least one additional orthologous species sequences is associated with wheat.

14. The computer assisted method of claim 1 wherein the performing a pairwise comparison between each pair of orthologous species sequences allows for one or variables to be used in the sequences.

15. A system for identifying regulatory elements, comprising:
a computer;
an article of software executing on the computer, the article of software adapted for performing steps of:
(a) receiving a first orthologous species sequence;
(b) receiving a word length;
(c) receiving a relative offset;
(d) receiving at least one additional orthologous species sequence, wherein each of the orthologous species sequences is associated with a species, and each of the species is an orthologous species;
(e) performing a pairwise comparison between each pair of orthologous species sequences;
(f) computing overlapping portions of the sequence overlapping the sequences of all of the orthologous species sequences within the relative offset and greater than or equal to the word length;
(g) providing an output to a user identifying the overlapping portions of the sequence for all of the orthologous species sequences to identify candidate regulatory elements.

16. The system of claim 15 wherein the candidate regulatory elements comprises a plurality of promoters.

17. The system of claim 15 wherein the receiving the word length comprises receiving a user-specified word length through a user interface associated with the article of software.

18. The system of claim 15 wherein the receiving the first orthologous species sequence comprises receiving a user-specified first orthologous species sequence through a user interface.

19. The system of claim 15 wherein the receiving the relative offset comprises receiving a user-specified relative offset through a user interface.

20. The system of claim 15 wherein the receiving the at least one additional orthologous species sequences include receiving the at least one additional orthologous species from a database.

21. The system of claim 15 wherein the first orthologous species sequence and the at least one additional orthologous species sequences are associated with plants.

22. The system of claim 21 wherein one of the first orthologous species sequence and the at least one additional orthologous species sequences being associated with maize.

23. The system of claim 21 wherein one of the first orthologous species sequence and the at least one additional orthologous species sequences being associated with soybeans.

24. The system of claim 21 wherein one of the first orthologous species sequence and the at least one additional orthologous species sequences being associated with wheat.

25. A computer-assisted method of identifying regulatory elements, comprising:
receiving a first sequence;
receiving a word length;
receiving a relative offset;
receiving at least one additional sequence;
performing a pairwise comparison between each pair of sequences;
computing using a computing device, overlapping portions of the first sequence overlapping the sequences of all of the sequences within the relative offset and greater than or equal to the word length;
providing an output to a user identifying the overlapping portions of the first sequence for all sequences to identify candidate regulatory elements.

26. The computer-assisted method of claim 25 wherein the first sequence and one or more of the at least one additional sequence are from a single species.

27. The computer-assisted method of claim 25 wherein the first sequence is from a first species and each of the at least one additional sequence are from species orthologous to the first species.

28. The computer-assisted method of claim 25 wherein the first sequence or at least one of the at least one additional sequence includes a variable.
